(19) United States
(12) Patent Application Publication
(10) Pub. No.: US 2010/0150807 A1
(43) Pub. Date: Jun. 17, 2010

(54) APPARATUS FOR PREPARING CA-P BIOMATERIAL BY PURIFICATION METHOD OF DIALYSIS AND SEPARATION

(75) Inventors: Shengmin Zhang, Wuhan (CN); Zhiye Chou, Wuhan (CN)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: HUAZHONG UNIVERSITY OF SCIENCE AND TECHNOLOGY, Wuhan, Hubei (CN)

(21) Appl. No.: 12/597,718

(22) PCT Filed: Apr. 22, 2008
(86) PCT No.: PCT/CN08/70769

§ 371 Date: Jan. 19, 2010

(30) Foreign Application Priority Data
Apr. 27, 2007 (CN) 200710051993.5

(51) Int. Cl. C01B 25/32; A61L 27/12; A61L 31/02; B01D 61/28; B01J 19/00

(52) U.S. Cl. 423/322; 210/321.71; 422/187; 422/109

(57) ABSTRACT

An apparatus for preparing Ca—P biomaterial by purification method of dialysis and separation is provided. The apparatus comprises a synthesis reactor(1), a purification module for dialysis and separation(2), a pure water tank(4), a product collection tank(5), a waste water tank(6), a chemical cleaning solution tank(7), two cleaning solution collection tanks(8-1, 8-2), a product transferring pump(9), a pure water transferring pump(10), a chemical cleaning solution transferring pump(11), a flow meter, a pressure meter, and pipelines connecting the above devices. The product and pure water form cross flow in the purification module for dialysis and separation via two passages for dialysis and purification. The chemical cleaning solution is supplied to the purification module for dialysis and separation via two pipelines for cleaning. The apparatus does not introduce any impurity into prepared Ca—P biomaterial, and phase change does not occur during purification process. Besides, the apparatus does not need traditional water washing and vacuum filtration devices, and has such advantages as high automatization, simple process, and controllable production cost.

### FIELD OF THE INVENTION

The present invention relates to the field of biomedical material, and more specific, to a producing apparatus for preparing Ca—P biomaterial by purification method of dialysis and separation, as well as a method of operating the same.

### BACKGROUND OF THE INVENTION

Nano Ca—P biomaterial mainly comprises nano hydroxyapatite, nano fluoro-substituted apatite, and modified apatites doped with strontium, zinc etc. These biomaterials are similar to the inorganic constituent of the human natural bone tissue, thus have good biocompatibility and bioactivity. Accordingly, such biomaterials are able to be used as repair materials and substitute materials for hard tissue. In addition, Ca—P biomaterial can also be used as separation materials and biocatalyst carrier for bioactive substances (such as, amino acid, polypeptide, protein etc.). Adhesive ability and proliferation ability of osteoblast can be significantly improved and then regeneration of bone tissue can be promoted by adding a little nano hydroxyapatite into other commonly synthesized biomaterials. Because of the excellent physical and chemical properties and biological properties, the preparation and application of nano Ca—P biomaterials has been a research hotspot and one of the important research subjects in the field of biomedical materials all over the world for many years.

However, at present, preparation methods for Ca—P biomaterials are restricted to production in lab-scale, and are not able to be applied to large-scale production. The reasons for which are the complex manual operations needed in the preparation process. The synthesis process and the purification process are disconnected, so manual operations are required. In the purification process, traditional washing and suctioning filtration is applied in a dead-end filtration manner and which resulting in too rapid thickening of the filter cake, thus only a small amount of slurry can be processed in a single filtration process, and the same operation must be repeated again and again until all the slurry is processed. In the centrifugal operation, because of the low sedimentation rate of nano particles, high speed or even ultra-high speed centrifuge, which is expensive and high work-consumption, as well as high skill required, is required. The above factors restrict the large-scale production of Ca—P biomaterials.

PRC patent application CN200610018593.X has disclosed a separation and purification process for preparing nano Ca—P biomaterial, that is, a purification method of dialysis and separation, which overcomes the mentioned drawbacks of the washing and suctioning filtration in traditional preparation of nano apatites, and is able to prepare non-sintered powders of nano Ca—P biomaterial with the a granularity of 50˜200 nm. The process is able to achieve automatic and continuous operations conveniently. However, at present, the dialysis purification process for preparing nano apatites is limited to lab study without corresponding producing apparatus and matched technological process.

### SUMMARY OF THE INVENTION

The primary objective of this invention is to provide a producing apparatus for preparing Ca—P biomaterials. By utilizing the producing apparatus, a continuous large-scale production of Ca—P biomaterial is achieved with high purity and recovery rate. Moreover, agglomeration will not present in the product during the production, and such producing apparatus has the characteristics of high automation, simple operation and economic energy consumption. To solve the mentioned technical problems, the present invention provides a producing apparatus for preparing Ca—P biomaterial by purification method of dialysis and separation. The apparatus comprises two main parts, that is, a preparation system and a purification system. The apparatus comprise a synthesis reactor with a temperature feedback regulation device and an pH value automatic regulation device, a group of purification modules for dialysis and separation for removing impurity ions in the initial product, a plurality of pressure adjustable transferring pumps and a plurality of matching valves, pressure meters and flow meters. Said purification module for dialysis and separation comprises one or more criteria-fixed standard purification elements. The purification elements can be connected in series to form a repeatedly purification module for dialysis and separation on the basis of the concentration of impurity ions in the initial product and/or the types of impurity ions to be removed in one hand, the purification elements also can be connected in parallel to form a multi-channel purification module for dialysis and separation on the basis of the quantity of production in another hand. Furthermore, by combining these two connection methods, the purification elements can be connected in series-parallel to form a purification module for dialysis and separation. The construction of the purification module is flexibly. Moreover, the construction and scale of the purification module can be designed independently according to specific production condition.

A producing apparatus for preparing Ca—P biomaterial by purification method of dialysis and separation according to the present invention (referring FIG. 1), comprises a synthesis reactor 1, a purification module for dialysis and separation 2, a pure water tank 4, a product collection tank 5, a waste water tank 6, a chemical cleaning solution tank 7, two cleaning solution collection tanks 8-1 and 8-2, a product transferring pump 9, a pure water transferring pump 10, a chemical cleaning solution transferring pump 11, a flow meter, a pressure meter, and pipelines connecting the above devices. The synthesis reactor 1, the product transferring pump 9, the product transferring pipeline 30, the purification module for dialysis and separation 2, the product collection pipeline 31 and the product collection tank 5 constitute a passage. The pure water tank 4, the pure water transferring pump 10, the main pipeline for pure water transferring 32, the purification module for dialysis and separation 2, the waste water collection pipeline 33 and the waste water tank 6 constitute another passage. The product and the pure water for cleaning form a cross-flowing purification in the standard purification elements 3 of the purification module for dialysis and separation 2 via two passages. The chemical cleaning solution tank 7 supplies chemical cleaning solution to the purification module for dialysis and separation 2 for cleaning via the chemical cleaning solution transferring pump 11 and two chemical cleaning pipelines 34 and 34-1.

The synthesis reactor 1 according to the present invention comprises a temperature feedback regulation device and a pH value automatic regulation device. The purification module for dialysis and separation 2 comprises one or more criteria-fixed standard purification elements 3, which are connected in series or in parallel or in parallel-series. The main part of each standard purification elements 3 is a cylinder with a length of 500˜1500 mm and a diameter of 50˜250 mm. The molecular weight cutoff of the standard purification elements 3 are the same or different, therefore the granularity ranges of the impurities to be removed are the same or different. In the case of a plurality of standard purification elements 3 being connected in series, impurities in the initial product can be removed from small to large in terms of the particle granularity by utilizing purification elements with different purification parameters at different series level. Each standard purification element 3 is vertically arranged on a stainless steel scaffold. On the shell of each standard purification element 3, there are four pipeline joints, wherein, the inlet and the outlet of the product are set at the lower end and upper end of the shell respectively, as well as the inlet and the outlet of the washing water are set at the lateral of the lower end and upper end of the shell respectively. The initial product slurry and the pure water for washing form a cross-flow in each standard purification elements 3.

In the device according to present invention, initial product and pure water for the cleaning form a cross-flow in the purification module for dialysis and separation, thus removing impurity ions and small amount of remaining high-molecular-weight molecules from the initial product slurry in a highly efficient and quick manner, so as to obtain Ca—P biomaterials with high purity and gets rid of manual batch-type operations in above mentioned traditional water washing and vacuum filtration process, and has the characteristics of high automation and simple operation. The purification process by using this apparatus does not need additional pressure for driving thus avoiding use of high-cost, high-energy-consumption and complex-operational devices, such as a vacuum filtration device and a high speed centrifuge, so as to efficiently decrease the production cost. The apparatus is therefore applicable for large-scale production of Ca—P biomaterials.

### BRIEF DESCRIPTION OF THE DRAWINGS

Further detail of the present invention will become evident from the attached drawings and operation manners.

FIG. 1 shows the connection of a producing apparatus for preparing Ca—P biomaterial by purification method of dialysis and separation according to the present invention;

FIG. 2 shows the pipeline connection of a single standard purification element 3;

FIG. 3 shows two series connection methods of standard purification elements in the purification module for dialysis and separation;

FIG. 4 shows a special-purpose series connection method of standard purification elements in the purification module for dialysis and separation.

### DETAILED DESCRIPTION OF THE PREFERRED EMBODIMENT

FIG. 1 shows the connection of a producing apparatus for preparing Ca—P biomaterial by purification method of dialysis and separation according to the present invention. The entire apparatus mainly comprises a synthesis reactor 1 and a purification module for dialysis and separation 2, and further comprises a pure water tank 4, a product collection tank 5, a waste water tank 6, a chemical cleaning solution tank 7, two cleaning solution collection tanks 8-1 and 8-2, as well as a transferring pump, a valve, a flow meter, a pressure meter and so on. The product transferring pump 9, the product transferring pipeline 30, the purification module for dialysis and separation 2, the product collection pipeline 31 and the product collection tank 5 constitute a passage for initial product in the synthesis reactor 1. The pure water tank 4, the pure water transferring pump 10, the main pipeline for pure water transferring 32, the purification module for dialysis and separation 2, the waste water collection pipeline 33 and the waste water tank 6 constitute another passage. The two passages form a cross-flow dialysis and complete purification process in the standard purification element 3 of the purification module for dialysis and separation 2 s. Final product is collected in the product collection tank 5 and waste water containing impurities is collected in the waste water tank 6. The chemical cleaning solution tank 7 supplies the chemical cleaning solution via the chemical cleaning solution transferring pump 11 and two chemical cleaning pipelines 34 and 34-1 to the purification module for dialysis and separation 2 for cleaning, so as to keep a high level of the purification efficiency and maintain the normal running of the apparatus. The connections of pipelines are controlled by butterfly valve to avoid conflict in each pipelines running.

For preparing a batch of Ca—P biomaterial, chemical materials (calcium salt, phosphate salt, etc.) react for 3˜5 hours in the synthesis reactor 1. Before initial product is taken out, the pure water tank 4 will be filled with sufficient pure water for cleaning and the pure water valve 14 and the waste water valve 15 will be opened while all other valves are closed, thus each standard purification element 3 in the purification module for dialysis and separation 2 is fully filled with pure water. Once pure water uniformly flows into the waste water tank 6, the initial product valve 12 and the final product valve 13 will be opened, and the initial product transferring pump 9 will be turned on. The initial product will be extracted from the push-out pipeline 29 by gas transferring method of using the pressure in the synthesis reactor 1. By observing the product pressure meter 21, the water pressure meter 23, the product flow meter 22 and the pure water flow meter 24, flow rates at the initial product valve 12 and the pure water valve 14 will be adjusted respectively on the basis of the production scale and the product concentration, so as to keep the flow rate of the pure water at the pure water valve 14 8˜15 times as the flow rate of product at the initial product valve 12. Keeping the working status until all the product is taken out from the synthesis reactor 1.

After all the initial product is taken out from the synthesis reactor 1 and the purification is completed, the initial product transferring pump 9, the initial product valve 12, the final product valve 13, the pure water transferring pump 10, the pure water valve 14 and the waste water valve 15 will be closed in turn. The purification module for dialysis and separation 2 will then stop working and the next production of Ca—P biomaterial may be started.

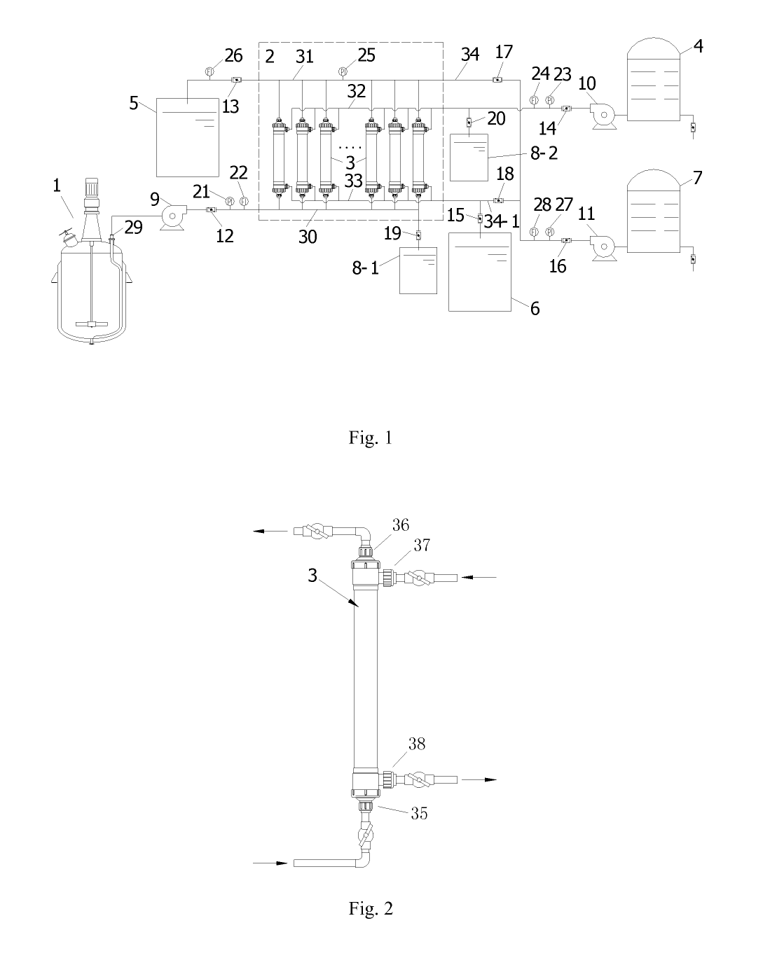

FIG. 2 shows the pipeline connection of a single standard purification element 3 in the preparation of Ca—P biomaterial by using purification method of dialysis and separation. FIG. 2 illustrates specific connection method of pipelines on the standard purification element 3. There are four pipeline joints on the standard purification element 3, wherein, the product inlet 35 and the product outlet 36 are connected in the element and set at the lower end and upper end of the shell respectively for the product slurry to flow through; the washing water inlet 37 and the washing water outlet 38 are connected in the element and set at the laterals of the lower end and upper end of the shell respectively for the washing water to flow. In the purification process, pure water for cleaning will firstly be pumped into the separation and purification element through the pipeline connected to the washing water inlet 37. After the washing water uniformly flows out from the washing water outlet 38, the initial product will then be pumped into purification element through the product inlet 35 slowly. For example, in a production process by using a standard purification element with a length of 1000 mm and a external diameter of 90 mm, DN25 pipeline joints will be employed on the shell of the standard purification element, flow rate of the washing water will be controlled within 500˜1000 ml/min and flow rate of the product slurry will be controlled within 50˜70 ml/min. Product slurry flows to the product collection pipeline through the product outlet 36 after the purification in the element and finally to the product collection tank 5.

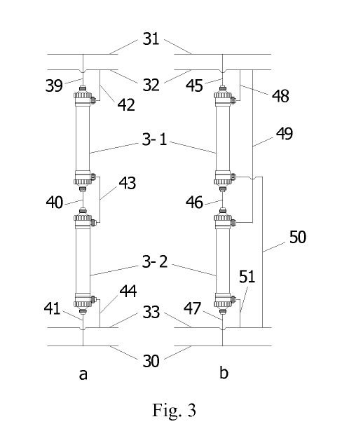

The purification module for dialysis and separation 2 shown in FIG. 1 may be constructed with a plurality of standard purification elements 3 connected in parallel to meet the need of the different scale of production. The purification module for dialysis and separation 2 may alternatively be constructed with a plurality of standard purification elements 3 connected in series as shown in FIG. 3. FIG. 3 shows two series connection methods of standard purification elements. In the series connection method, the initial product slurry taken out from the synthesis reactor 1 flow into the standard purification element 3-2 and 3-1 via the product pipeline 30 for dialysis purification, and finally flow into the product collection pipeline 31. In FIG. 3, method a and b are two different series connection methods for washing water pipelines. In the series connection method a, washing water flows into the upper standard purification element 3-1 via the washing water branch pipeline 42 and then flows into the lower standard purification element 3-2 via the connection pipeline 43, finally flows into the waste water collection pipeline 33 via the waste water branch pipeline 44. This series connection is equivalent to prolong the purification time. In the series connection method b, washing water flows into the upper standard purification element 3-1 and the lower standard purification element 3-2 via the washing water branch pipelines 48 and 49 respectively, and then flows into the waste water collection pipeline 33 via the waste water branch pipelines 50 and 51 respectively. This series connection is equivalent to prolong washing time and increase washing water consumption. These two series connection methods are applicable to the purification for initial production with high concentration. Series connections include but not limited to two-stage and pipeline connection methods also include but not limited to the two methods shown in FIG. 3, series connections can also be applied to three or more stage series and corresponding pipeline connection methods.

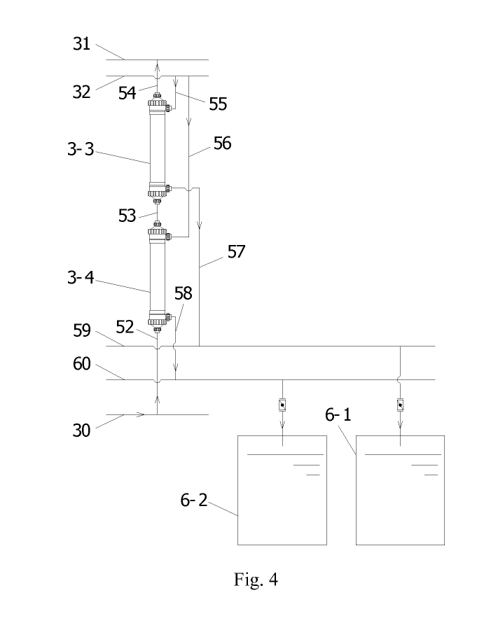

FIG. 4 shows a special-purpose series connection method of standard purification elements in the purification module for dialysis and separation. For example, in the two-stage series connection purification shown in FIG. 4, reference 3-3 is a standard purification element with high molecular weight cut off for removing organic impurities with high molecular weight, reference 3-4 is a standard purification element with low molecular weight cut off for removing impurity ions with low molecular weight. The initial product taken out from the synthesis reactor 1 firstly flows into the element 3-4 via the product pipeline 30 to remove low molecular weight impurity ions by dialysis purification, while high molecular weight organic impurities remain in the product slurry. Product slurry then flows into the element 3-3 to remove high molecular impurities, and finally flows into the product collection pipeline 31. Pure water for cleaning flows into series-connected elements 3-3 and 3-4 via the washing water branch pipelines 55 and 56 respectively and then flows out from the elements via the waste water branch pipelines 57 and 58. In this series connection purification method, waste water discharged from the elements 3-3 and 3-4 flows into two separate waste water collection pipelines 59 and 60 respectively, and will finally be collected in two waste water collection tanks 6-1 and 6-2. In this series connection, two waste water collection tanks 6-1 and 6-2 replace the single waste water collection tank 6 illustrated in FIG. 1, thus good for the environment and generating maximum economic benefits. This special-purpose series connection method includes but not limited to two-stage taken as the example, but also can be applied to three or more stage series connection with this special purpose.

The chemical cleaning system shown in FIG. 1 shares most of pipelines with the purification module for dialysis and separation 2, so that these pipelines will be cleaned during the cleaning of standard purification element 3 and ensuring free-flowing in the pipelines. Time interval for chemical cleaning may be one to four weeks according to type and concentration of the product slurries. Chemical cleaning steps comprise three steps of acid washing, alkaline washing and reverse osmosis water washing. A typical formulation of acid cleaning solution is aqueous solution of citric acid with pH 2; a typical formulation of alkaline cleaning solution is aqueous solution of sodium hydroxide and sodium hypochlorite with pH 12; reverse osmosis water will be used in reverse osmosis water washing During the chemical cleaning, the initial product valve 12, the final product valve 13, the pure water valve 14 and the waste water valve 15 is closed, and the main valve for cleaning solutions 16, the cleaning solution valves 17 and 18 and the cleaning solution collection valves 19 and 20 is open. Since the product slurry in the production is alkaline, a acid washing will thus be carried out first, which comprising: filling acid cleaning solution into the chemical cleaning solution tank 7, opening the chemical cleaning solution pump 11, and regulating the main valve for cleaning solutions 16 until the chemical cleaning solution pressure meter 27 reaches 0.2 MPa. The acid washing lasts for 30 min. Waste cleaning solution will be collected in the cleaning solution collection tanks 8-1 and 8-2. The chemical cleaning solution pump 11 and the valve 16 will be closed in turn after the acid washing. An alkaline washing will then be carried out, which comprising: filling alkaline cleaning solution into the chemical cleaning solution tank 7, opening the chemical cleaning solution pump 11, and regulating the main valve for cleaning solutions 16 until the chemical cleaning solution pressure meter 27 reaches 0.2 MPa. The alkaline washing lasts for 30 min. Waste cleaning solution will be collected in the cleaning solution collection tanks 8-1 and 8-2. The chemical cleaning solution pump 11 and the valve 16 will be closed in turn after the alkaline washing. At last, a reverse osmosis water washing will be carried out, which comprising: replacing the chemical cleaning solution tank 7 with a special reverse osmosis water tank filled with sufficient reverse osmosis water, opening the pump 11, regulating the valve 16 until the pressure meter 27 reaches 0.2 MPa, detecting pH value of the water discharged from the element 3, and terminating reverse osmosis water washing once pH of discharged water reaches 7. The chemical cleaning solution pump 11 and the valves 16, 17, 18, 19 and 20 will be closed after the reverse osmosis water washing.

The valves mentioned above may be a butterfly valve, a gate valve or a globe valve.

1. An apparatus for preparing Ca—P biomaterial by purification method of dialysis and separation, characterized in that,
the apparatus comprising a purification module for dialysis and separation (2), a product transferring pipeline (30), a product collection pipeline (31), a main pipeline for pure water transferring (32) and a waste water collection pipeline (33);
wherein, said purification module for dialysis and separation (2) comprises one or more standard purification modules (3), interior of the standard purification modules (3) is divided into a product slurry channel and a washing water channel by a membrane structure;
the product transferring pipeline (30), the product slurry channel of the purification module for dialysis and separation (2), and the product collection pipeline (31) constitute a first passage; the main pipeline for pure water transferring (32), the washing water channel of the purification module for dialysis and separation (2), and the waste water collection pipeline (33) constitute a second passage; product slurry and the washing water get to the purification module for dialysis and separation (2) respectively through these first and second passages and form a flow-dialysis to exchange substance through the membrane structure of the standard purification modules (3).

2. The apparatus according to claim 1, characterized in that, when the purification module for dialysis and separation (2) comprises more than one standard purification elements (3), these standard purification elements (3) are connected in series, in parallel or in series-parallel.

3. The apparatus according to claim 1, characterized in that, the apparatus further comprises a chemical cleaning solution tank (7) with a chemical cleaning solution transferring pump (11), a first chemical cleaning solution pipeline (34), a second chemical cleaning solution pipeline (34-1), a first cleaning solution collection tank (8-1) and a second cleaning solution collection tank (8-2); wherein, the chemical cleaning solution tank (7), the first chemical cleaning solution pipeline (34), the product collection pipeline (31), the purification module for dialysis and separation (2), the product transferring pipeline (30) and the first cleaning solution collection tank (8-1) constitute a third passage; the chemical cleaning solution tank (7), the second chemical cleaning solution pipeline (34-1), the waste water collection pipeline (33), the purification module for dialysis and separation (2), the main pipeline for pure water transferring (32) and the second cleaning solution collection tank (8-2) constitute a fourth passages; chemical cleaning solutions wash the purification module for dialysis and separation (2) and portions of pipelines through the third and fourth passages.

4. The apparatus according to claim 1, characterized in that the apparatus further comprises a synthesis reactor (1) connected to the product transferring pipeline (30) for preparing the product slurry, a pure water tank (4) connected to the main pipeline for pure water transferring (32) for providing pure water, a product collection tank (5) connected to the product collection pipeline (31) for collecting the product, and a waste water tank (6) connected to the waste water collection pipeline (33) for collecting the waste water.

5. The apparatus according to claim 4, characterized in that said synthesis reactor (1) is provided with an initial product valve (12), said pure water tank is provided with a pure water valve (14), said product collection tank (5) is provided with a final product collection valve (13) and said waste water tank (6) is provided with a waste water valve (15).

6. The apparatus according to claim 3, characterized in that said chemical cleaning solution tank (7) is provided with a main chemical cleaning solutions valve (16), a first chemical cleaning solution valve (17) is provided between the main chemical cleaning solutions valve (16) and said first chemical cleaning solution pipeline (34), a second chemical cleaning solution valve (18) is provided between the main chemical cleaning solutions valve (16) and said second chemical cleaning solution pipeline (34-1), said first cleaning solution collection tank (8-1) and a second cleaning solution collection tank (8-2) are provided with a first cleaning solution collection valve (19) and a second cleaning solution collection valve (20) respectively.

7. The apparatus according to claim 4, characterized in that said synthesis reactor (1) is provided with a product transferring pump (9) and the pure water tank (4) is provided with a pure water transferring pump (10).

8. The apparatus according to claim 4, characterized in that a product pressure meter (21) and a product flow meter (22) are provided near said initial product valve (12), meanwhile a pure water pressure meter (23) and a pure water flow meter (24) are provided near said pure water valve (14); the synthesis reactor (1) is provided with a temperature feedback regulation device and an pH value automatic regulation device.

9. The apparatus according to claim 1, characterized in that the granularity ranges or the molecular weight cut off of the membrane structure in each standard purification element (3) are the same or different.

10. The apparatus according to claim 1, characterized in that when a plurality of standard purification elements (3) are connected in series in the production, standard purification elements (3) with different parameters are employed in different stage to remove impurities from small to large in turn in terms of granularity or molecular weight.

11. The apparatus according to claim 1, characterized in that the flow rate of the pure water at the pure water valve (14) should be kept as 8˜15 times as the flow rate of product at the initial product valve (12).

12. An apparatus for preparing Ca—P biomaterial by purification method of dialysis and separation, comprising a synthesis reactor (1), a purification module for dialysis and separation (2), a pure water tank (4), a product collection tank (5), a waste water tank (6), a product transferring pipeline (30), a product collection pipeline (31), a main pure water transferring pipeline (32) and a waste water collection pipeline (33);
characterized in that,
said purification module for dialysis and separation (2) comprises one or more standard purification modules (3), interior of the standard purification modules (3) is divided into a product slurry channel and a washing water channel by a membrane structure; each of the standard purification modules (3) has a product inlet (35) and a washing water outlet (38) setting at the lower end of the shell, as well as a product outlet (36) and a washing water inlet (37) setting at the upper end of the shell;
the synthesis reactor (1), the product transferring pipeline (30), the product inlet (35) of the purification module for dialysis and separation (2), the product slurry channel of the purification module for dialysis and separation (2), the product outlet (36) of the purification module for dialysis and separation (2), the product collection pipeline (31), and the product collection tank (5) constitute a first passage; the pure water tank (4), the main pure water transferring pipeline (32), the washing water inlet (37) of the purification module for dialysis and separation (2), the washing water channel of the purification module for dialysis and separation (2), the washing water outlet (38) of the purification module for dialysis and separation (2), the waste water collection pipeline (33) and the waste water tank (6) constitute a second passage; the product slurry and the washing water get to the purification module for dialysis and separation (2) through these first and second passages, and form a flow-dialysis to exchange substance through the membrane structure of the standard purification modules (3).

13. A method for preparing Ca—P biomaterial by purification method of dialysis and separation, characterized in that the method comprises the following steps:
1) fill pure water for washing into a washing water channel of a standard purification element (3) of a purification module for dialysis and separation (2);
2) when pure water keep flowing out of the standard purification element (3) of the purification module for dialysis and separation (2), fill the product slurry flows into a product slurry channel of the standard purification element (3) of the purification module for dialysis and separation (2), so that the product slurry and the washing water form a flow-dialysis to exchange substance through a membrane structure of the standard purification modules (3);
3) collect the product from the standard purification element (3) of the purification module for dialysis and separation (2).

14. The method according to claim 13, characterized in that the method further comprises the following steps:
1) chemical materials react for 3˜5 hours in a synthesis reactor (1);
2) before initial product is taken out, fill sufficient pure water into a pure water tank (4), and open a pure water valve (14) installed on the pure water tank (4) and a waste water valve (15) installed on a waste water tank (6), run a pure water transferring pump (10) installed on the pure water tank (4) so as to fill pure water to each standard purification element (3) in the purification module for dialysis and separation (2);
3) once pure water uniformly flows into the waste water tank (6), open an initial product valve (12) installed on the synthesis reactor (1) and the final product valve (13) installed on a product collection tank (5), run an initial product transferring pump (9) installed on the synthesis reactor (1) to take initial product slurry out from the synthesis reactor (1), the product slurry flows into the product collection tank (5) after the being purified in the purification module for dialysis and separation (2), then keep the working status until all the product is taken out from the synthesis reactor (1);
4) once all the initial product is taken out from the synthesis reactor (1), shut down the initial product transferring pump (9) and close the initial product valve (12) and the final product valve (13), then shut down the pure water transferring pump (10) and close the pure water valve (14) and the waste water valve (15) in turn.

15. The method according to claim 14, characterized in that during the preparation, adjust the initial product valve (12) and the pure water valve (14) on the basis of the detection values obtain from a product pressure meter (21) and a product flow meter (22) installed near the initial product valve (12), as well as a water pressure meter (23) and a pure water flow meter (24) installed near the pure water valve (14) for proper flow rates on the basis of the production scale and the product concentration, so as to keep the flow rate of the pure water at the pure water valve (14) as 8˜15 times as the flow rate of product at the initial product valve (12).

16. The method according to claim 15, characterized in that the production method further comprises a chemical cleaning process with following steps:
1) close the initial product valve (12), the final product valve (13), the pure water valve (14) and the waste water valve (15), and open a main chemical cleaning solutions valve (16) installed on a chemical cleaning solution tank (7), a first chemical cleaning solution valve (17) installed between the main chemical cleaning solutions valve (16) and a first chemical cleaning solution pipeline (34), a second chemical cleaning solution valve (18) installed between the main chemical cleaning solutions valve (16) and a second chemical cleaning solution pipeline (34-1), and a first cleaning solution collection valve (19) installed on a first cleaning solution collection tank (8-1) and a second cleaning solution collection valve (20) installed on a second cleaning solution collection tank (8-2);
2) fill sufficient acid cleaning solution into the chemical cleaning solution tank (7), open a chemical cleaning solution pump (11) installed on the chemical cleaning solution tank (7), and keep acid washing for at least 30 min, then close the chemical cleaning solution pump (11) and the main chemical cleaning solutions valve (16) in turn;
3) fill sufficient alkaline cleaning solution into the chemical cleaning solution tank (7), open the main chemical cleaning solutions valve (16) and the chemical cleaning solution pump (11) in turn, and keep alkaline washing for at least 30 min, then close the chemical cleaning solution pump (11) and the main chemical cleaning solutions valve (16) in turn;
4) replace the chemical cleaning solution tank 7 with a special reverse osmosis water tank filled with sufficient reverse osmosis water, open the main chemical cleaning solutions valve (16) and the chemical cleaning solution pump (11) in turn, monitor pH value of the water discharged from the standard purification element (3), and then terminate reverse osmosis water washing once pH of discharged water reaches 7;
5) close the chemical cleaning solution pump (11) and the main chemical cleaning solutions valve (16), the first chemical cleaning solution valve (17), the second chemical cleaning solution valve (18), the first cleaning solution collection valve (19) and the second cleaning solution collection valve (20).
